SEQUENCE LISTING (16 sequences)

SEQ ID NO: 1 - protein, 145 aa, Homo sapiens
MEFGLSWLFLVAILKGVQCEVQLLESGGGLVQPGGSLRLSCAASGFTFSSYAMSWIRQAPGKGLEWVSAISASGGSTYYADSVKGRFTISRDNSKNTLYLQMNSLRAEDTAVYYCAKDRIVGATNYYYGMDVWGQGTTVTVSSAS

SEQ ID NO: 2 - protein, 130 aa, Homo sapiens
MDMRVPAQLLGLLLLWFPGSRCDIQMTQSPPSVSASVGDRVTITCRASQGISSWLAWYQHKPGKAPKLLIYGASSLQSGVPSRFSGSGSGTDFTLTISSLQPEDFATYYCQQANSFPITFGQGTRLEIKR

SEQ ID NO: 3 - protein, 149 aa, Homo sapiens
MDLMCKKMKHLWFFLLLVAAPRWVLSQLQVQESGPGLVKPSETLSLICTVSGGSIRTSGYYWGWFRQPPGKGLEWIGTSHNSGSTYYNPSLKSRVTISVDTSKNQFSLKLNSVTAADTAVYYCARQGYDFKVNIDVWGQGTTVTVSSAS

SEQ ID NO: 4 - protein, 128 aa, Homo sapiens
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKR

SEQ ID NO: 5 - protein, 142 aa, Homo sapiens
MEFGLSWVFLVALLRGVQCQVQLVESGGGVVQPGRSLRLSCAASGFTFSYYGMHWVRQAPGKGLEWVAVIWYDGSNKYYADSVKGRFTISRDNSKKTLYLQMNSLRAEDTAVYYCARDGHSSGWGDFQHWGQGTLVTVSSAS

SEQ ID NO: 6 - protein, 128 aa, Homo sapiens
MEAPAQLLFLLLLWLPDTTGEIVLTQSPATLSLSPGERATLSCRASQSVSSSLAWYQQKPGQAPRLLIYDASNRATGIPARFSGSGSGTDFTLTISSLEPEDFAIYYCQQRSNWPPTFGQGTKVEIKR

SEQ ID NO: 7 - protein, 142 aa, Homo sapiens
MKHLWFFLLLVAAPRWVLSQLQLQESGPGLVKPSETLSLTCTVSGGSISTSSYYWGWIRQPPGKGLEWIGTIYYNGSTYYSPSLKSRVSISVDTSKNQFSLKLSSVTAADTSVYYCARQGYDIKINIDVWGQGTTVTVSSAS

SEQ ID NO: 8 - protein, 128 aa, Homo sapiens
MEAPAQLLFLLLLWLPDTTGEIVLTQSPATLSLSPGERATLSCRASQSVSSYLAWYQQKPGQAPRLLIYVASNRATGIPARFSGSGSGTDFTLTISSLEPEDFAVYYCQQRSNWPLTFGGGTKVEIKR

SEQ ID NO: 9 - protein, 142 aa, Homo sapiens
MEFGLSWVFLVALLRGVQCQVQLVESGGGVVQPGRSLRLSCAASGFTFSHYGMHWVRQAPGKGLEWVALIWYDGSNKYYADSVKGRFTISRDNSKNTLYLQMNSLRAEDTAVYYCARDPLAAGTSYFDYWGQGTLVTVSSAS

SEQ ID NO: 10 - protein, 126 aa, Homo sapiens
MSPSQLIGFLLLWVPASRGEIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIK

SEQ ID NO: 11 - protein, 142 aa, Homo sapiens
MEFGLSWLFLVAILKGVQCEVQLLESGGGLVQPGGSLRLSCAASGFTFSSYAMTWVRQAPGKGLEWVSDISGSGGYTYYADSVKGRFTISRDNSKNTLYLQMNSLRAEDTAVYYCAKTGAGSGSYSPDSWGQGTLVTVSSAS

SEQ ID NO: 12 - protein, 130 aa, Homo sapiens
MDMRVLAQLLGLLLLCFPGARCDIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKR

SEQ ID NO: 13 - protein, 142 aa, Homo sapiens
MEFGLSWLFLVAILKGVQCEVQLLESGGGLVQPGGSLRLSCAASGFTFSSYAMSWVRQAPGKGLEWVSDISGSGGYTYYADSVKGRFTISRDNSKNTLYLQMNSLRAEDTAVYYCAKTGDGSGSYSPDSWGQGTLVTVSSAS

SEQ ID NO: 14 - protein, 130 aa, Homo sapiens
MDMRVLAQLLGLLLLCFPGARCDIQMTQSPSSLSASVGDRVTITCRASQGISRWLAWYQQKPEKAPKSLIYAASSLQSGVPSRFSGSGSGTDFTLTISSLQPEDFATYYCQQYNSYPYTFGQGTKLEIKR

SEQ ID NO: 15 - protein, 142 aa, Homo sapiens
MEFGLSWLFLVAILKGVQCEVQLLESGGGLVQPGGSLRLSCAASGFTFSSYAMSWVRQAPGKGLEWVSDISGSGGYTYYADSVKGRFTISRDNSKKTLYLQMNSLRAEDTAVYYCAKTGDGSGSYSPDYWGQGTLVTVSSAS

SEQ ID NO: 16 - protein, 130 aa, Homo sapiens; identical to SEQ ID NO: 12

(19) United States
(12) Patent Application Publication
(10) Pub. No.: US 2012/0020989 A1
(43) Pub. Date: Jan. 26, 2012

(54) METHODS OF TREATING COLORECTAL CANCER

(75) Inventors: Eri TAGUCHI, Shizuoka (JP); Tomohiro SUDO, Tokyo (JP); Hironori KANDA, Tokyo (JP)

(73) Assignee: KYOWA HAKKO KIRIN CO., LTD., Tokyo (JP)

(21) Appl. No.: 13/009,193

(22) Filed: Jan. 19, 2011

Related U.S. Application Data
(60) Provisional application No. 61/296,180, filed on Jan. 19, 2010.

(51) Int. Cl. A61K 39/395; A61P 35/00

(52) U.S. Cl. 424/174.1

(57) ABSTRACT

The present invention provides a pharmaceutical composition and a treating method for colorectal cancer which is difficult for a surgical treatment, comprising an anti-A33 human antibody and a chemotherapy agent in combination. Based on the present invention, a pharmaceutical composition and a treating method comprising the combination of anti-A33 human antibody with CPT-11 (irinotecan) can exhibit a certain degree of antitumor effect on a patient with colorectal cancer who is found recurrence after previously-initiated therapy and/or a patient with colorectal cancer who is difficult to be cured by existing treatments for colorectal cancer.

### BACKGROUND OF THE INVENTION

1. Field of the Invention

This invention relates to a useful therapeutic pharmaceutical composition and a method for treating patients having progressed colorectal cancer, comprising using a human antibody which binds to A33 antigen and a chemotherapeutic agent in combination.

2. Brief Description of the Background Art

Colorectal cancer is the third-leading cancer type in the world, and the colorectal cancer occupied 7.9% of the world cancer-related deaths in 2000. Since in Japan, the cancer is the first leading cause of death and the number of the patients has been increasing every year, the development of an agent and therapeutic method which are highly effective and safe is strongly desired.

Five years survival rate of colorectal cancer is relatively high with stage I and stage II since lesions of the cancer can be removed almost completely by surgical operation. However, the five years survival rate is low under such as conditions that the cancer is progressed (stage III and stage IV).

For the patients with stage III colorectal cancer, when the cancer can be excised, the recurrence after excision is the serious problem. In the patients diagnosed as colorectal cancer, about 50% of the recurrence is found despite that cancer tissues remaining after the excision are not observed.

Accordingly, an assisted chemotherapy (adjuvant therapy) is carried out after the operation for the purpose of preventing recurrence. As a typical assisted chemotherapy, FOLFOX or capecitabine therapy is used.

However, since an anti-tumor agent is administrated for a long period, the patients suffer from nerve toxicity, blood toxicity, diarrhea, nausea and the like as its side effects. Though the chemotherapy is carried out for the patients who cannot undergo excision, the results including recurrence are not sufficient yet.

Accordingly, the unmet needs at stage III are lowering of recurrence rate (improvement of refractory disease survival period leads to the prolongation of survival period), reduction of side effects, more convenient administration method (therapeutic method) and further lower cost.

Since it is almost impossible to remove lesions of the cancer by surgical operation (radical surgery), in the case of the colorectal cancer patients of stage IV, a chemotherapy (or a radiation therapy) is carried out for the purpose of prolongation the survival period.

Although a treatment by a 5-FU preparation alone was the main method until the middle of 1990, multi-drug combination therapies have been developed one after another along with the development of novel chemotherapeutic agents. Since irinotecan, oxaliplatin, bevacizumab, cetuximab, panitumumab and the like were approved, various therapeutic choices are present in the current therapeutic method depending on the situations.

As the main standard treatment, there are many cases in which the FOLFOX therapy or combination use of the FOLFOX therapy with bevacizumab is used as the first line therapy. There are many cases where the patients who became refractory to a first line therapy use a treatment comprising the FOLFIRI therapy or the FOLFOX therapy and bevacizumab in combination as the second selection.

There is also a case in which the FOLFIRI therapy is used as a choice of first line therapy, and in this case, the FOLFOX therapy is used as the second line in many cases.

Most of the third line therapy is combination therapy of irinotecan and cetuximab. However, it cannot become a choice for the patients having a background that there is a mutation in the K-ras gene.

For the patients who is not administrated with an EFGr inhibitor as the second line and is not tolerance to the irinotecan, cetuximab alone or panitumumab alone is used as the third line in many cases.

The medical needs which have not been satisfied for patients at stage IV include prolongation of surviving period, reduction of side effects, more convenient administration method (therapeutic method) and further lower cost.

As an antibody against A33 antigen which is a class I cell membrane protein called “A33”, one of the Ig super family and an antitumor specific antigen, and a mouse anti-A33 antibody, a humanized anti-A33 antibody, a human A33 antibody and the like have been reported (see Patent Literatures 1 to 4 and Non-patent Literatures 1 to 5).

It is known that the above antigen relates to colorectal cancer and gastric cancer (see Patent Literature 2, Patent Literature 3 and Non-patent Literature 6).

In addition, a phase I clinical trial on colon cancer patients using the humanized A33 antibody was carried out in recent years (see Non-patent Literatures 4 and 5). In the former report on the antibody, a partial response was found in one of 11 patients who could be administrated with an antibody.

Moreover, according to a report relating to a trial using the humanized A33 antibody and a chemotherapy (Non-patent Literature 5), a partial response was found in three of 12 patients who could be administrated with an antibody, and a mixed response was found in one patient.

However, the humanized A33 antibody showed a considerably high tumor reaction in the phase I clinical trials as described in the above, but a human anti-humanized antibody (namely “HAHA”) was produced in both of the trials at a high probability of 50% or more. Interestingly, the HAHA was not found in the patients who showed high tumor reactivity.

In general, it is known that when a non-human antibody such as a mouse antibody is administered to human, it is recognized as a foreign substance and a human antibody against a mouse antibody (human anti-mouse antibody, “HAMA”) is induced in the human body.

It is known that the HAMA reacts with the administered mouse antibody and thereby causes side effects (Non-patent Literatures 7 to 10), quickens disappearance of the mouse antibody from the body (Non-patent Literatures 11 to 13) and lowers therapeutic effect of the mouse antibody (Non-patent Literatures 14 and 15).

Accordingly, an antibody which does not induce HAHA and HAMA is desired in the therapy.

### CITATION LIST

### Patent Literature

[Patent Literature 1] U.S. Pat. No. 5,958,412
[Patent Literature 2] U.S. Pat. No. 5,643,550
[Patent Literature 3] U.S. Pat. No. 5,160,723
[Patent Literature 4] WO2006/028197

### Non-Patent Literature

[Non-patent Literature 1] KING D. J. et al., British J. Cancer (1995) 72, 1364-1372
[Non-patent Literature 2] Welt S. et al., J. Clinical Oncology (1994), 12, 1561-1571
[Non-patent Literature 3] Welt S. et al., J. Clinical Oncology (1996), 14, 1787-1797
[Non-patent Literature 4] Welt S. et al., Clinical Cancer Res. (2003), 9, 1338-1346
[Non-patent Literature 5] Welt S. et al., Clinical Cancer Res. (2003), 9, 1347-1353
[Non-patent Literature 6] Garin-Chesa P. G. et al., international J. Oncology (1996), 9, 465-471
[Non-patent Literature 7] Current Opinion in Immunology 17, 275 (2005)
[Non-patent Literature 8] J Immunol., 141, 21 (1988)
[Non-patent Literature 9] Eur J Immunol., 22, 447 (1992)
[Non-patent Literature 10] Proc. Natl. Acad. Sci., 94.6346 (1997)
[Non-patent Literature 11] Leukemia and Lymphoma., 43, 1855 (2002)
[Non-patent Literature 12] Hum. Pathol., 38, 564 (2007)
[Non-patent Literature 13] Hum. Pathol., 36, 886 (2005)
[Non-patent Literature 14] FEBS Lett., 579, 6179 (2005)
[Non-patent Literature 15] Cancer Res., 65, 7378 (2005)
[Non-patent Literature 16] Hum. Pathol., 36, 886 (2005)
[Non-patent Literature 17] Oncogene, 13, 2328 (2006)
[Non-patent Literature 18] Virchows Arch., 448, 52 (2006)
[Non-patent Literature 19] J. Immunol., 135, 1530 (1985)
[Non-patent Literature 20] Cancer Res., 46, 6489 (1986)

### SUMMARY OF THE INVENTION

As described in the above, a new therapeutic method and an effective therapeutic method are required for the colorectal cancer patients who show resistance to the conventional colorectal cancer treatment.

Therefore, an object of the invention is to provide a method from which a certain clinical effect can be expected for the patients who show resistance to the conventional treatment of colorectal cancer, by the combination using a human A33 antibody which does not induce HAHA and a chemotherapy agent in combination.

The present invention can provide a therapeutic pharmaceutical composition and a therapeutic method in which disappearance/reduction/stabilization of tumor, and/or improvement of clinical symptoms accompanied with colorectal cancer for the patients who show resistance to the conventional treatment of colorectal cancer can be expected.

### BRIEF DESCRIPTION OF THE DRAWINGS

FIG. 1(A)(a) to FIG. 1(A)(c) show effects of combination use of N26 antibody (KRN330) and a chemotherapy agent in SCID mouse. FIG. 1(A)(a) to FIG. 1(A)(c) show effects of combination use of N26 antibody and CPT-11. In FIG. 1(A)(c), shows a result of comparison in survival curves by Log-rank test. Also, ** is p<0.01, and n.s. shows that there is no significant difference.

FIG. 1(B)(a) to FIG. 1(B)(c) show effects of combination use of N26 antibody (KRN330) and a chemotherapy agent in SCID mouse. FIG. 1(B)(a) to FIG. 1(B)(c) show effects of combination use of N26 antibody and 5-FU. In FIG. 1(B)(c), shows a result of comparison in survival curves by Log-rank test. Also, n.s. shows that there is no significant difference.

FIG. 2(a) and FIG. 2(b) show effects of combination use of N26 antibody (KRN330) and 5-FU or oxaliplatin in nude rat. The numerical value in parenthesis shows drug dose (mg/kg).

FIG. 3(a) and FIG. 3(b) show effects of combination use of N26 antibody (KRN330) and CPT-11 or Avastin in nude rat. The numerical value in parenthesis shows drug dose (mg/kg).

### DETAILED DESCRIPTION OF THE INVENTION

This invention relates to the following 1 to 22.

1. A pharmaceutical composition for treating colorectal cancer, comprising a combination of effective amounts of an anti-A33 human antibody and irinotecan.
2. The pharmaceutical composition for treating colorectal cancer described in the above item 1, for simultaneously or separately administering the anti-A33 human antibody and the irinotecan.
3. The pharmaceutical composition for treating colorectal cancer described in the above item 1 or 2, wherein the anti-A33 human antibody and the irinotecan are comprised in a single preparation.
4. The pharmaceutical composition for treating colorectal cancer described in any one of the above items 1 to 3, wherein each of the anti-A33 human antibody and the irinotecan are comprised in a separate preparation.
5. The pharmaceutical composition for treating colorectal cancer described in any one of the above items 1 to 4, wherein the colorectal cancer is at least one of the recurrent and refractory colorectal cancers.
6. The pharmaceutical composition for treating colorectal cancer described in any one of the above items 2 to 5, wherein the anti-A33 human antibody is administered every week or every other week.
7. The pharmaceutical composition for treating colorectal cancer described in any one of the above items 2 to 6, wherein the anti-A33 human antibody is at a dose of from 0.1 mg/kg to 3 mg/kg.
8. The pharmaceutical for treating colorectal cancer described in any one of the above items 2 to 7, wherein the irinotecan is at a dose of 180 mg/kg.
9. The pharmaceutical composition for treating colorectal cancer described in any one of the above items 1 to 8, wherein the patient with colorectal cancer is a colorectal cancer patient who has not previously received treatment with cetuximab which is an antibody against an epithelial cell growth factor receptor.
10. The pharmaceutical composition for treating colorectal cancer described in any one of the above items 1 to 9, wherein the patient with colorectal cancer is a patient with a colorectal cancer who has a mutation in K-ras gene.
11. A method, comprising administering effective amounts of an anti-A33 human antibody and the irinotecan in combination to a patient with colorectal cancer.
12. The method described in the above item 11, wherein the anti-A33 human antibody and the irinotecan are administered simultaneously or separately.
13. The method described in the above item 12, wherein the anti-A33 human antibody and the irinotecan are comprised in a single preparation.
14. The method described in any one of the above items 11 to 13, wherein each of the anti-A33 human antibody and the irinotecan is comprised in a separate preparation.
15. The method described in any one of the above items 11 to 14, wherein the colorectal cancer is at least one of recurrent colorectal cancer and refractory colorectal cancer.
16. The method described in any one of the above items 11 to 15, wherein the anti-A33 human antibody is administered every week or every other week.
17. The method described in any one of the above items 11 to 16, wherein the anti-A33 human antibody is administered at a dose of from 0.1 mg/kg to 3 mg/kg.
18. The method described in any one of the above items 11 to 17, wherein the irinotecan is administered at a dose of 180 mg/kg.
19. The pharmaceutical composition for treating colorectal cancer described in any one of the above items 11 to 18, wherein the patient with colorectal cancer is a colorectal cancer patient who has not received previous treatment with cetuximab which is an antibody against an epithelial cell growth factor receptor.
20. The method described in any one of the above items 11 to 19, wherein the patient with colorectal cancer is a patient with a colorectal cancer who has a mutation in K-ras gene.
21. The method described in any one of the above items 11 to 20 wherein the method is a method for treating a patient with colorectal cancer.
22. Use for the manufacture of a pharmaceutical composition for treating colorectal cancer, comprising a combination of effective amounts of an anti-A33 human antibody and irinotecan.

This invention relates to a pharmaceutical composition for treating colorectal cancer, comprising a combination of effective amounts of an anti-A33 human antibody and irinotecan. Also, the invention relates to a therapeutic method, which comprises administering effective amounts of an anti-A33 human antibody and irinotecan simultaneously or separately to a patient with colorectal cancer.

Colorectal cancer is one of cancer species which occurs in the large intestine (cecum, colon and rectum), and those which occur in the anal canal are included therein. In general, the colorectal cancer is divided into cecum cancer, colon cancer and rectum cancer. In the invention, the colorectal cancer means a cancer including these cancer species.

As the colorectal cancer patient in the invention, a patient with at least one of recurrent colorectal cancer and refractory colorectal cancer is preferable, and a patient who has received a chemotherapy used for colorectal cancer is more preferable.

Examples of the colorectal cancer in the invention include a patient who obtains no effect despite of having received a treatment with the above-mentioned combination of a chemotherapy and a therapeutic antibody, such as a combination use therapy of 5-FU (5-fluorouracil) and LV (leucovorin), CPT-11 (irinotecan), oxaliplatin (L-OHP), capecitabine, TS-1, FOLFIRI therapy (combination use of 5-FU, LV and CPT-11), FOLFOX therapy (combination use of 5-FU, LV and L-OHP), XELOX therapy (combination use of capecitabine and L-OHP), bevacizumab, cetuximab, or a combination use of the FOLFIRI therapy (combination use of 5-FU, LV and CPT-11) and bevacizumab, combination use of the XELOX therapy and bevacizumab, or who has received these treatments but has a relapse.

In addition, as the colorectal cancer in the invention, preferred are a colorectal cancer patient who did not undergo pretreatment with an antitumor antibody having antibody-dependent cellular cytotoxicity (ADCC) activity as ae main drug effect and a colorectal cancer patient who has a mutation in the K-ras gene. Examples of the antitumor antibody having ADCC activity as a main drug effect include cetuximab (an epithelial cell growth factor receptor (EGFR) antibody) and the like.

The first selection of colorectal cancer treatment is a surgical therapy. When the colorectal cancer is local, this is the best therapy for aiming at a radical cure. However, there is a case in which a surgical therapy cannot be carried out due to the disease stage of each patient and various medical factors, and in that case, a chemotherapy and a radiotherapy and an multimodality therapy in which these therapeutic methods are combined (The Journal of the National Comprehensive Cancernetwork, Volume 7, Number 8, September 2009).

It is possible to prepare the anti-A33 human antibody to be used in the invention using the A33 protein disclosed in WO97/08189 as the antigen and by a commonly known human antibody preparing technique [e.g., see Kuroiwa et al., Nat. Biotechnol., 20: 889 (2002); WO 98/24893; WO 92/01047; WO 96/34096; WO 96/33735; U.S. Pat. No. 5,413,923; U.S. Pat. No. 5,625,126; U.S. Pat. No. 5,633,415; U.S. Pat. No. 5,569,825; U.S. Pat. No. 5,661,016; U.S. Pat. No. 5,545,806; U.S. Pat. No. 5,814,318; U.S. Pat. No. 5,885,792; U.S. Pat. No. 5,916,771 and U.S. Pat. No. 5,939,598].

In addition, as an anti-A33 human antibody, the antibody disclosed in WO2006/028197 can also be used. Examples include the anti-A33 human antibodies produced by hybridomas M10 (depositary number: FERM BP-10107), M96 (depositary number: FERM BP-10108), M165 (depositary number: FERM BP-10106), N26 (depositary number: FERM BP-10109), Q47 (depositary number: FERM BP-10104), Q54 (depositary number: FERM BP-10105) or R5 (depositary number: FERM BP-10103) [each of these hybridomas has deposited on Aug. 24, 2004, in International Patent Organism Depositary, National Institute of Advanced Industrial Science and Technology (Central 6, 1-1-1 Higashi, Tsukuba, Ibaraki, Japan)], anti-A33 human antibodies which recognize the same epitopes recognized by the antibodies produced by these hybridomas, or anti-A33 human antibodies which comprises a heavy chain variable region and a light chain variable region comprising the same sequences of the heavy chain variable region and a light chain variable region, respectively, of the anti-A33 human antibody produced by one of these hybridomas or functional fragments thereof.

In addition, examples include an anti-A33 human antibody comprising the heavy chain variable region represented by SEQ ID NO:23 and the light chain variable region represented by SEQ ID NO:25, an anti-A33 human antibody comprising the heavy chain variable region represented by SEQ ID NO:27 and light chain variable region represented by SEQ ID NO:29, an anti-A33 human antibody comprising the heavy chain variable region represented by SEQ ID NO:31 and the light chain variable region represented by SEQ ID NO:33, an anti-A33 human antibody comprising the heavy chain variable region represented by SEQ ID NO:35 and the light chain variable region represented by SEQ ID NO:37, an anti-A33 human antibody comprising the heavy chain variable region represented by SEQ ID NO:39 and the light chain variable region represented by SEQ ID NO:41, an anti-A33 human antibody comprising the heavy chain variable region represented by SEQ ID NO:43 and the light chain variable region represented by SEQ ID NO:45, an anti-A33 human antibody comprising the heavy chain variable region represented by SEQ ID NO:47 and the light chain variable region represented by SEQ ID NO:49 or an anti-A33 human antibody comprising the heavy chain variable region represented by SEQ ID NO:51 and the light chain variable region represented by SEQ ID NO:53, which has been disclosed in WO2006/028197, or functional fragments thereof.

The “functional fragment” means a part of an antibody (partial fragment) which maintains at least one of the actions of the antibody to antigen, and, specifically, examples include F(ab′)₂, Fab′, Fab, Fv, disulfide linked Fv, single chain Fv (scFv), polymers thereof and the like [D. J. King, Applications and Engineering of Monoclonal Antibodies, 1998, T. J. International Ltd.].

Alternatively, the “functional fragment” is a fragment of an antibody and is a fragment which can bind to an antigen.

In addition, examples of the “anti-A33 human antibody” also include an antibody which specifically attacks an A33-expressing tumor cell via an immune system by ADCC activity (antibody-dependent cellular cytotoxicity) and CDC activity (complement-dependent cytotoxicity).

As the class of the aforementioned antibody, IgG is preferable. In addition, as the subclass, human IgG1, human IgG2, human IgG3 and human IgG4 are preferable, and IgG1 is more preferable.

As the method for enhancing the ability of ADCC activity and CDC activity, conventionally known methods can be used. Examples of the method for controlling the effector activity include a method for controlling a sugar chain bound to Fc region of an antibody, a method for introducing amino acid modification for substituting an amino acid residue of Fc region of an antibody, and the like.

Examples of the method for controlling a sugar chain bound to Fc region of an antibody include a method for lowering ADCC activity or CDC activity by removing a sugar chain at position 297 of an IgG antibody [Molecular Immunology, 32, 1311 (1995), WO2008/030564], a method for lowering CDC activity by reducing binding of galactose to an Fc region of an antibody, and the like.

In addition, examples of the method for controlling a sugar chain bound to the antibody Fc region include a method for producing an antibody which comprises a sugar chain in which fucose is not bound to the base part of N-acetylglucosamine (GlcNAc) to which the sugar chain is bound, in an N-linked sugar chain bound to asparagine at position 297 of Fc region of an IgG antibody, (U.S. Pat. No. 7,214,775 and U.S. Pat. No. 6,946,292), a method for producing an antibody comprising a sugar chain to which bisecting GlcNAc is bound [Nature Biotechnology, 17, 176 (1999)], a method for producing an antibody comprising a sugar chain to which a galactose (Gal) which is bound to a non-reducing end is bound [Hum. Antibod. Hybridomas, 5, 143-151 (1994)] and the like.

As the method for modifying an amino acid residue of the Fc region of the antibody, examples include a method in which the effector activity is controlled by carrying out amino acid modification of the Fc region of the antibody (J. B. C., 277, 26733-26740, 2002, U.S. Pat. No. 6,737,056, U.S. Pat. No. 7,297,775, U.S. Patent Application No. 2007/002060, WO2005/070963), a method in which the effector activity is controlled by carrying out domain exchange between respective subclasses of the Fc region of the antibody (WO2007/011041) and the like.

When an antibody is produced using recombinant DNA techniques, the antibody is formed in cells or cell membrane periphery voids or directly secreted into the medium. In the former case, it is necessary to remove host cells or cell lysis fragments by centrifugation or ultrafiltration. In the latter case, the cell waste can be separated by centrifugation. In addition, it can be concentrated using a protein concentration filter.

The antibody composition prepared from the cells can be purified using affinity chromatography, hydroxyapatite chromatography or gel electrophoresis. As the ligand of the affinity chromatography, protein A can be used.

In addition, the antibody having a high purity which is required as a therapeutic preparation can be obtained by combining with ion exchange chromatography which uses an anionic or cationic ion exchange resin.

The therapeutic preparation of the anti-A33 human antibody to be used in the invention is prepared for storage in a form of a freeze-dried preparation or an aqueous solution by mixing a pharmacologically acceptable carrier, adjuvant or a stabilizer having optional components with the antibody having a desired purity.

An appropriate amount of pharmaceutically acceptable salt is used in the carrier in order to make the preparation isotonic. The acceptable carrier, adjuvant or stabilizer is nontoxic for the cells at the dose and concentration to be used and examples include a buffer solution such as of phosphoric acid, citric acid, glutamic acid, arginic acid and other organic acids; an antioxidant including ascorbic acid and methionine; an antiseptic (e.g., octadecyldimethylbenzylammonium chloride; hexamethonium chloride; benzalkonium chloride; benzethonium chloride; phenol, butyl and benzyl alcohol; alkyl parabens, such as methyl and propyl paraben; cathecol; resorcinol; cyclohexanol; 3-pentanol; and m-cresol); a polypeptide having a low molecular weight (the number of residues is less than 10); a protein such as serum albumin, gelatin and immunoglobulin; a hydrophilic polymer such as polyvinyl pyrrolidone; an amino acid such as glycine, glutamine, asparagine, histidine, arginine or lysine; a monosaccharide and a disaccharide such as glucose, mannose or dextrin and other carbohydrate; a chelating agent such as EDTA; a saccharide such as sucrose, mannitol, trehalose or sorbitol; a salt-forming counter ion such as sodium; a metal complex (e.g., a Zn-protein complex); and/or a nonionic surfactant such as TWEEN, PLURONICS or polyethylene glycol (PEG).

Dose of the anti-A33 human antibody may be any amount as long as it is an effective amount. As a standard, it is preferably from 0.1 mg/kg to 10 mg/kg, more preferably from 0.1 mg/kg to 3 mg/kg, further preferably from 0.5 mg/kg to 1 mg/kg per once per subject (including an adult patient). It can be optionally increased or decreased depending on symptoms of the subject (including an adult patient) and using amount of other therapeutic agents.

The term “effective amount” means the amount of a drug effective for treating disease or malady of a mammal. In a case of a cancer, by administering a therapeutically effective amount of a drug, the number of cancer cells is reduced; size of the tumor becomes small; infiltration of cancer cells into peripheral organs is inhibited (namely, a certain degree of delay is preferable and suspension is more preferable); metastasis of the tumor is inhibited (namely, a certain degree of delay is preferable and suspension is more preferable); growth of the tumor is inhibited to a certain degree; and/or one or more symptoms relating a cancer are reduced to a certain degree. When the drug prevents growth of the cancer cells and/or perishes the existing cancer cells, it could be cell growth inhibitory and/or cell toxicity.

In the treatment of a cancer, the in vivo effect can be measured, for example, by evaluating survival period, time to progression (TTP), response rate (RR), duration of response, and/or quality of life. The response rate (RR) means total of the rate of complete response and partial response.

Examples of the irinotecan to be used in the invention include an anti-malignant tumor agent synthesized from camptothecin which is an antitumor alkaloid derived from Camptotheca acuminata Decne, and it inhibits DNA synthesis by inhibiting type I DNA topoisomerase. The cell killing effect is specific to the S phase of the cell cycle, and it is an agent which exhibits its effect in a limited time manner.

Examples of the irinotecan preparation to be used in the invention include CRT-11 (manufactured by YAKULT HONSHA CO., LTD.) and the like.

As the dose of the irinotecan, it may be any amount as long as it is an effective amount, but is preferably from 50 mg/m² to 350 mg/m², more preferably from 100 mg/m² to 200 mg/m², further preferably 180 mg/m².

The pharmaceutical composition of the invention is a pharmaceutical composition for the treatment of colorectal cancer comprising a combination of anti-A33 human antibody and the irinotecan, and it is preferable that the anti-A33 human antibody and the irinotecan are administrated simultaneously or separately.

According to the method of the invention, the administration of an anti-A33 human antibody and the irinotecan simultaneously or separately means that two agents are administered to a patient simultaneously or separately by the following administration method and administration interval.

The simultaneous administration means that both of the active components are administered simultaneously, namely instantaneously, or essentially at the same time. Also, the separate administration means that the first active component is firstly administered, and after a certain period of time, the second active component is administered. The certain period of time is preferably from 30 minutes to 12 hours.

When the anti-A33 human antibody and the irinotecan are simultaneously administered, both of the active components may be present in a single preparation or each active component may be present in a separate preparation. In addition, when the anti-A33 human antibody and the irinotecan are separately administered, each active component is present in a separate preparation.

That is, the pharmaceutical composition of the invention may be used as a single preparation comprising both of the anti-A33 human antibody and the irinotecan, or may be used as separate preparations in which one preparation comprises the anti-A33 human antibody and the other preparation comprises the irinotecan.

Sometimes one of the active components must be administered at a higher frequency than the other active component which requires to be administered once a day, for example twice a day of administration. Accordingly, a method in which the first active component and the other ingredients are administered in combination or sequentially, and after a certain period of time, the other active component alone is administered again, and the reverse method, are also included in the simultaneous or separate administration.

Accordingly, the pharmaceutical composition of the invention also include a pharmaceutical composition which comprising as separate preparations in which one of the preparations comprising an anti-A33 human antibody and irinotecan and the other preparation comprising either an anti-A33 human antibody or irinotecan.

As the administration method of the invention, intravenous injection is preferable and intravenous drip infusion is more preferable. The intravenous drip infusion means that introduction of a drug into a vein of an animal or human patient is carried out for approximately 5 minutes or longer, preferably for approximately from 30 minutes to 90 minutes.

As the administration interval, administration in every week (each week), every other week (every two weeks) or every three weeks can be considered, but such as the necessity of setting of a drug withdrawal depending on taking symptoms of the subject (including adult patient) and use amount of other therapeutic agents can be optionally judged.

Also, in the administration method of the invention, a chemotherapy used for the treatment of colorectal cancer can be combined with the above-mentioned two preparations.

Specific examples of the chemotherapy used for the treatment of colorectal cancer include a combination use therapy of 5-FU (5-fluorouracil) and LV (leucovorin), CPT-11 (irinotecan), oxaliplatin (L-OHP), capecitabine, TS-1, IFL or FOLFIRI therapy (combination use of 5-FU, LV and CPT-11), FOLFOX therapy (combination use of 5-FU, LV and L-OHP), XELOX therapy (combination use of capecitabine and L-OHP) and the like.

In addition to the two preparations to be administered in the invention, a therapeutic antibody can also be administered. Examples of the therapeutic antibody include bevacizumab, cetuximab, avastin and the like. In addition, it is also possible to combine the therapeutic antibody with the above-mentioned chemotherapy.

In the efficacy evaluation, the standard of RECIST (response evaluation criteria in solid tumors) (J. National Cancer Institute, 2000, Vol. 92, No. 3, 205-216) can be used.

That is, the complete response (CR) means disappearance of all of the target lesions. The partial response (PR) means that at least 30% decrease in the sum of the longest diameter of target lesions, taking as reference the baseline sum of the longest diameter.

The progress (progressive disease; PD) means that a 20% or greater increase in the sum of the longest diameter of target lesions, taking as reference the smallest sum of the longest diameter recorded during and after the start of the treatment.

The stable (stable disease; SD) means that the shrinkage of tumor is insufficient to be regarded as PR and the enlargement of tumor is also insufficient to be regarded as PD, in comparison with the total of the smallest maximum major axes in and after the start of the treatment.

Next, the invention is specifically described based on examples, but the invention is not limited to the following examples.

### EXAMPLES

### I. Inspection by Non-Clinical Models

### Example 1

### Inspection by SCID mouse

(1) SCID mouse and colorectal cancer cell line

SCID mice (C.B-17/Icr-scid, female, 6-weeks old) were purchased from CLEA Japan Inc. and reared in an SPF (specific pathogen free) equipment.

A colorectal cancer-derived cell line LS174T (purchased from ATCC) was used as the tumor cell and cultured using RPMI 1640 medium (manufactured by GIBCO, Invitrogen) containing 10% FBS (manufactured by HyClone) and 1% penicillin-streptomycin (manufactured by GIBCO, Invitrogen).

A xenograft model was prepared by intraperitoneally transplanting the cultured LS174T cell into the SCID mouse at a dose of 1×10⁷ cells/200 μl/head.

### (2) Agents and Administration Protocol

An anti-A33 human antibody (KRN330) (hereinafter referred to as N26 antibody) comprising the heavy chain variable region represented by SEQ ID NO:23 and the light chain variable region represented by SEQ ID NO:25 was used as an anti-A33 human antibody.

5-FU (5FU Injection 250 Kyowa) was obtained from Kyowa Hakko Kirin Co., Ltd. and CPT-11 (Campto (registered trademark) Injection or Irinotecan Hydrochloride Injection) was obtained from YAKULT HONSHA CO., LTD. In the control group, a dilution medium of the anti-A33 human antibody (10 mM of sodium L-glutamate, 262 mM of D-sorbitol and 0.05 mg/ml of polysorbate 80, pH 5.5) was used.

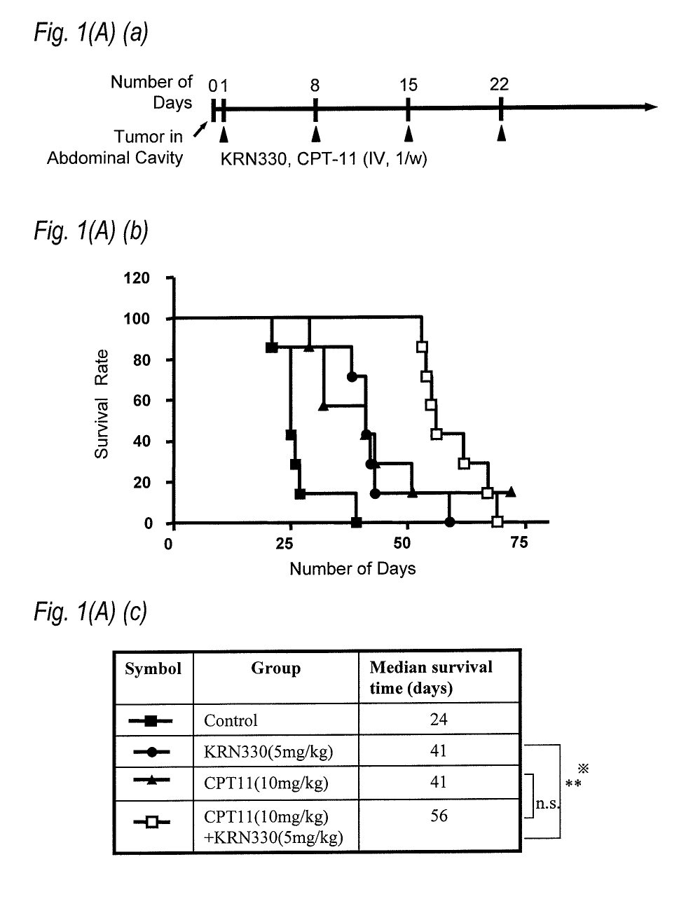

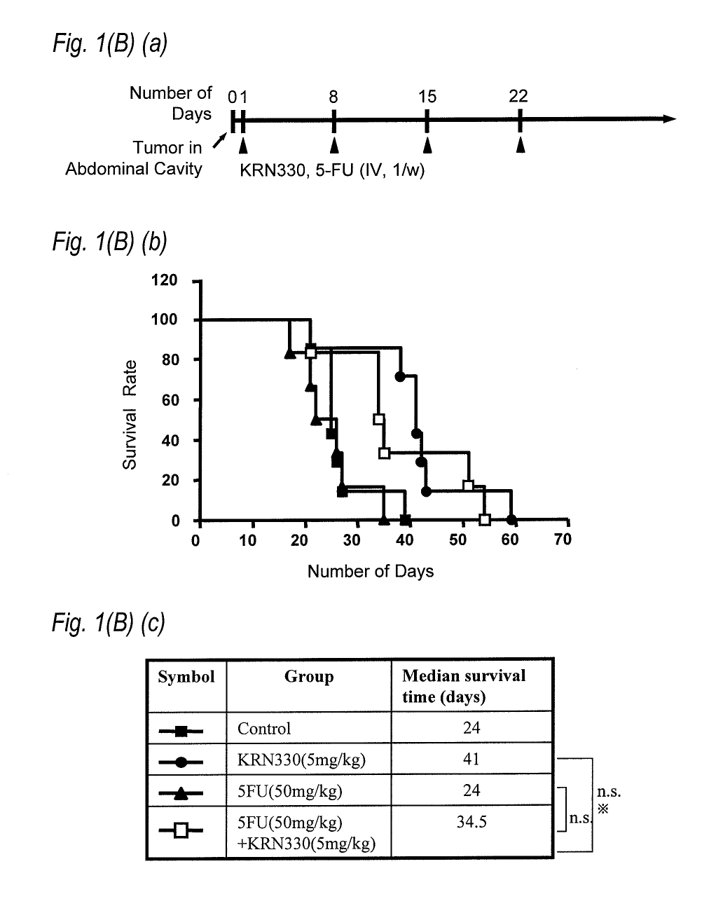

Administration schedules of agents are shown in FIG. (A)(a) and FIG. 1(B)(a), respectively. After transplanting the cultured LS174 cell into the abdominal cavity of SCID mice on day 0, their body weights were measured on the next day of the transplantation (on day 1) to divide them into six groups in such a manner that the average body weight of each group became equal (A, B, E and F groups=seven animals per group, C and D groups=six animals per group).

The grouping day was regarded as the initial administration day, and thereafter, 5 mg/kg of an anti-A33 human antibody, 10 mg/kg of CPT-11 (irinotecan) or 50 mg/kg of 5-FU was administered into the mouse caudal vein at a dose of 10 ml/kg, four times (on day 1, day 8, day 15 and day 22) at a frequency of once a week, and the surviving period was monitored. On the same day of the drug administration, an adjuvant was intravenously administered to the control group.

### (3) Results

Survival curve of each administration group was plotted by the Kaplan-Meier method and the survival curves of each group were compared using log-rank test, with the results shown in FIG. 1(A)(b) and FIG. 1(A)(c) and FIG. 1(B)(b) and FIG. 1(B)(c). In this connection, though the results of FIG. 1(A)(b) and FIG. 1(A)(c) and FIG. 1(B)(b), and FIG. 1(B)(c) are separately shown, these were carried out in the same test, and results of the control group and the N26 antibody (5 mg/kg) group in FIG. 1(A)(b) and FIG. 1(A)(c) are identical to those in FIG. 1(B)(b) and FIG. 1(B)(c).

As shown in FIG. 1(A)(b) to FIG. 1(A)(c) and FIG. 1(B)(b) to FIG. 1(B)(c), significant prolongation of surviving period was found in both of the groups administrated N26 antibody or CPT-11 alone in comparison with the group administrated the adjuvant alone. In addition, the surviving period was significantly prolonged in the group of the N26 antibody and CPT-11 in combination in comparison with the adjuvant group and the N26 antibody alone group.

As shown in FIG. 1(A)(c), regarding the surviving period median (median survival time: MST) of each group, the adjuvant administration group, N26 antibody single preparation administration group, CPT-11 single preparation administration group and N26 antibody and CPT-11 combination use administration group were 24 days, 41 days, 41 days and 56 days, respectively.

On the other hand, as shown in FIG. 1(B)(c), prolongation of surviving period was not found in the 5-FU alone administration group of this study, and each of the median survival times of the 5-FU alone administration group and the adjuvant administration group was 24 days. In addition, despite that significant prolongation of surviving period was found in the N26 antibody alone administration group, significant prolongation of surviving period was not found in the 5-FU in combination with the N26 antibody administration group, in comparison with the adjuvant administration group.

### Example 2

### Inspection Using Nude Rat

### (1) Nude Rat and Colorectal Cancer Cell Line

Nude rat (F344/NJcl-rnu/rnu, female, 8 weeks old) was purchased from CLEA Japan Inc. and reared in an SPF equipment. LS174T was used as the tumor cell in the same manner as in Example 1. A gallbladder cancer model was prepared by subcutaneously grafting with the cultured LS174T cell at a dose of 5×10⁶ cells/100 μl/head.

### (2) Agents and Administration Protocol

In the same manner as Example 1, N26 antibody was used as an anti-A33 human antibody. In addition, 5-FU and CPT-11 are used in the same manner as Example 1. Oxaliplatin (ELPLAT (registered trademark) For Injection or Oxaliplatin For Injection) was obtained from YAKULT HONSHA CO., LTD., and Avastin (registered trademark) (bevacizumab) was obtained from Genentech Inc.

Administration schedule of each agent was carried out in the following manner. When the size of tumor became from 30 m³ to 50 m³ after subcutaneously grafting with LS174T cell to the nude rat, grouping was carried out so as to be five animals per group in such a manner that the tumor volume of each group became uniform (on day 0).

Each of N26 antibody (5 mg/kg) and 5-FU (100 mg/kg), oxaliplatin (15 mg/kg), CPT-11 (10 mg/kg) and avastin (5 mg/kg) was administered as a single preparation or a combination use with N26 antibody from the caudal vein four times every other week (on day 0, day 7 day 14 or day 21) at a dose of 5 ml/kg.

The adjuvant was intravenously administered to the control group in the same manner. The tumor volume of each individual was measured at a frequency of once or twice a week.

### (3) Results

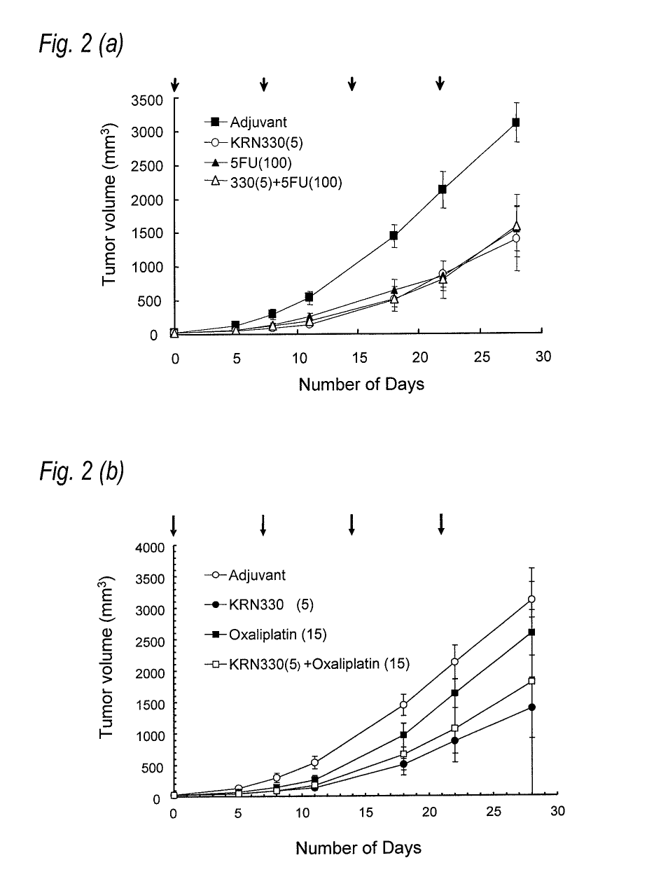

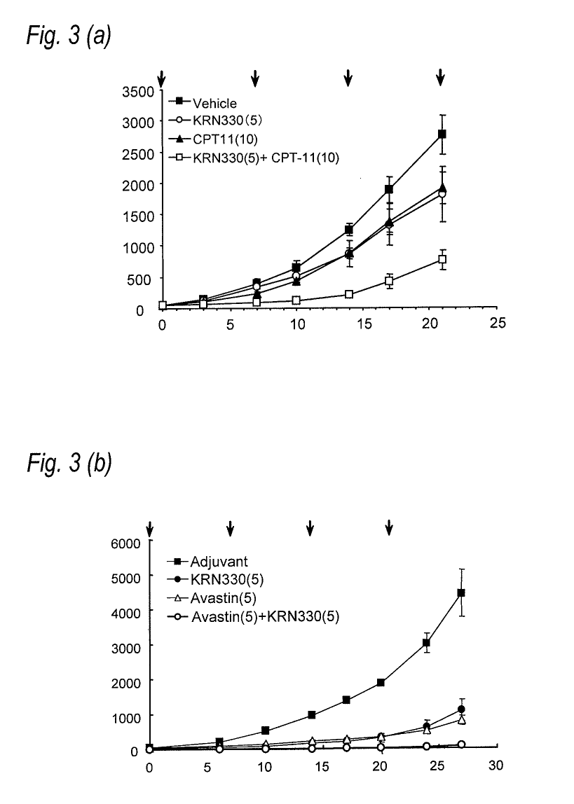

Each test results is shown in FIG. 2(a) to FIG. 2(b) and FIG. 3(a) to FIG. 3(b). As shown in FIG. 2(a), both of N26 antibody and 5FU showed significant antitumor effect by single administration on the nude rat LS174T subcutaneous xenograft model. However, enhancement of the antitumor effect by the combination use of N26 antibody and 5FU was not found.

On the other hand, as shown in FIG. 2(b), the single administration showed a slight antitumor effect in the oxaliplatin administration group but enhancement of the antitumor effect by its combination use of oxaliplatin with N26 antibody was not found, but rather, antitumor effect of the combination use group was lower than that of the anti-A33 antibody single administration group. In addition, significant body weight loss was also found in the oxaliplatin administration group.

The effect of combination use of N26 antibody with CPT-11 and effect of its combination use with avastin and the results are shown in FIG. 3(a) to FIG. 3(b). As shown in FIG. 3(a), the same level of antitumor effect as the control group was found in the anti-A33 human antibody single administration group and the CPT-11 single administration group. On the other hand, in the groups in which these were used and administered in combination, significant antitumor effect was found and enhancement of antitumor effect by their combination use was shown.

In addition, as shown in FIG. 3(b), in the test in which effect of combination use with avastin was examined, both of the anti-A33 antibody single preparation administration and avastin single preparation exhibited similar degree of significant antitumor effect, and the further enhanced antitumor effect was found by administering them in combination.

Based on the above, enhancement of antitumor effect by administrating was confirmed for only CPT-11 and avastin in combination with the anti-A33 human antibody in the nude rat. Similar to the results of SCID mouse in Example 1, enhanced antitumor effect of the combination use of the anti-A33 human antibody with 5-FU was not able to confirm. In addition, it was confirmed that combination use of the anti-A33 human antibody with oxaliplatin rather had an adverse effect.

Based on these results, it was suggested that N26 antibody and CPT-11 (irinotecan) was a good combination when N26 antibody was administrated in combination with chemotherapy agents.

### II. Clinical Test

### Example 3

### (1) Single Agent Phase I Clinical Trial of N26 Antibody

Using the same N26 antibody in Examples 1 and 2, such as safety and efficacy of six doses (0.1, 0.3, 1, 3, 6, 10 mg/kg) of N26 antibody on patients with progressive colorectal cancer who had no other standard therapeutic methods were examined in accordance with an administration schedule in which N26 antibody was administered once a week for four weeks, or every other week for eight weeks, each four times in total.

The number of patients to whom the N26 antibody was administered once or more was six in the 0.1 mg/kg administration group, four in the 0.3 mg/kg administration group, four in the 1 mg/kg administration group, twenty in the 3 mg/kg administration group, two in the 6 mg/kg administration group and two in the 10 mg/kg administration group (38 in total).

In this connection, regarding the patients who wished continuous administration of N26 antibody, they were able to be transferred to the continuous administration when certain standards of safety and efficacy were satisfied.

In the N26 antibody administration groups at a dose of 6 and 10 mg/kg, grade 3 gastrointestinal disorders (diarrhea, spastic abdominal pain and vomiting) were found in two patients in each group.

Expression of A33 which is the antigen of N26 antibody is found not only in cancer cells such as colorectal cancer but also in normal intestinal cells. Accordingly, such a phenomenon was considered as a biological activity of N26 antibody caused by specific and excess binding of N26 antibody to intestinal cells including normal cells due to the administration of excess dose of N26 antibody.

On the other hand, in 34 patients administered with 3 mg/kg or less of N26 antibody, dose limiting toxicities including gastrointestinal disorders were not found at all under such conditions that an appropriate pre-administration was carried out so as to diminish gastrointestinal disorders. Therefore, it was shown that the maximum tolerated dose was 3 mg/kg.

Further importantly, it was reported that when a humanized antibody against the same antigen (huA33) was administered to human, a human anti-humanized antibody which caused an unpreferable influence upon its safety and efficacy was produced at a high frequency [Cancer Res., 61: 6851-6859 (2001)]. However, in the case of N26 antibody, production of the human anti-humanized antibody was not found at all in the all cases throughout the entire study period including the continuous administration period.

Among 32 patients which were possible to be evaluated, 47% (15 patients) of the patients was stable (Stable Disease/unchangeable) as the best response. During the N26 antibody administration period, the patients in which the cancer was not progressed over a long period were relatively frequent, and for example, the patients who was stable more than 20 weeks were 7 patients (47%) in total of these 15 patients (found at 4 doses of 0.1, 0.3, 1 and 3 mg/kg).

Further, progress of the cancer was not found over a very long period in three of these patients. The progress of the cancer was not found for 39 weeks in one of them, and for one year or more in the other two patients. The treating period of N26 antibody of these three patients was the longest among any of the standard treatments as a pretreatment.

In addition, interestingly, a considerable reduction of 50% or more of a tumor marker CEA which was considered due to the antitumor effect of N26 antibody was found in three of the 15 patients who were stable as the best response. The changes in CEA in each of patients were as follows.

One is a patient who was administered with 3 mg/kg of N26 antibody, and the CEA value on day 35 after the start of the N26 antibody administration (66.3 ng/ml) was reduced by 63% in comparison with the value before the N26 antibody administration (179 ng/ml).

Another is a patient who was administered with 1 mg/kg of N26 antibody and exhibited no progress of the cancer for 39 weeks, and the CEA value on day 218 after the start of the N26 antibody administration (7.4 ng/ml) was reduced by 59% in comparison with the value before the N26 antibody administration (18.1 ng/ml).

The other is a patient who was administered with 3 mg/kg and 1 mg/kg of N26 antibody and exhibited no progress of the cancer for one year or more, and the CEA value on day 232 after the start of the N26 antibody administration (116.2 ng/ml) was reduced by 90% in comparison with the value before the N26 antibody administration (1,127 ng/ml).

Thus, based on the above, since there were facts that the cancer did not progress over a very long period in relatively many patients, significant reduction of a tumor marker, CEA and the like, it was considered that the N26 antibody single preparation is effective on the progressive colorectal cancer patients who do not respond to the standard therapeutic method.

Based on these, a phase I/II clinical trial of N26 antibody and irinotecan in combination was carried out as the next test of the single agent phase I clinical trial of the N26 antibody.

### Example 4

### (2) Phase I/II Clinical Trial of N26 Antibody and Irinotecan in Combination

### (Patients to be Tested)

The patients capable of participating in this test were mainly the patients who 1) were 18 years old or older, 2) had metastatic colorectal cancer which could be measured and was histologically confirmed, 3) developed recurrence or exhibited progress in three or less of pretreatments including the FOLFOX therapy (combination use of fluorouracil, oxaliplatin and leucovorin), the CapOx therapy (combination use of capecitabine and oxaliplatin), combination use of the FOLFOX therapy and bevacizumab and combination use of the CapOx therapy and bevacizumab and 4) passed at least 4 weeks from a chemotherapy, a radiotherapy, an immunotherapy, a biological therapy, other investigational drugs and a main surgical treatment.

On the other hand, the patients who were not able to participate in this test were mainly the patients who (1) have a grade 2 or more of allergy reaction or past history of oversensitivity by the administration of a humanized or human antibody, (2) have a past history of the production of a human anti-humanized antibody or human anti-human antibody or (3) underwent treatment of the anti-A33 antibody.

### (Abstract of Test Operation Plan)

In this test, patients of metastatic colorectal cancer who developed recurrence or exhibited progress in three or less of pretreatments including the FOLFOX therapy, CapOx therapy, combination use of the therapy and bevacizumab and combination use of the CapOx therapy and bevacizumab were used as the objects, and safety, efficacy and the like of N26 antibody (starting dose: 1 mg/kg) were examined by an administration schedule of administering it once a week or once a two weeks and the irinotecan (180 mg/m²) every two weeks, each for 6 weeks.

In this connection, regarding the patients who wished to receive continuous administration of N26 antibody, it was possible to shift to the continuous administration when there was no concern about safety and no progression of the cancer. Since it is known that N26 antibody and the irinotecan cause specific gastrointestinal disorders when these agents were used as single preparations, respectively, there is a possibility to exhibit a more severe phenomenon when both of the agents are used in combination.

Accordingly, the main object of the first phase part of the phase I/II clinical trial of combination use was to confirm safety of the combination therapy of N26 antibody and the irinotecan.

### (Treating Method and Results)

The number of patients to whom N26 antibody was administered once or more was four patients in the 1 mg/kg administration group (once every two weeks of N26 antibody administration) and 15 patients in the 0.5 mg/kg administration group (8 patients for once every weeks of N26 antibody administration and 7 patients for once a week of N26 antibody administration) (19 in total).

In the 1 mg/kg of N26 antibody and the irinotecan administration groups, pyrogenic neutronpenia of grade 3 or 4 (two patients), diarrhea (two patients), colitis (one patient), dehydration (one patient), hypopotassiumemia (one patient) and hypoalbuminemia (one patient) were found in three of the 4 patients.

In accordance with the clinical protocol, the dose of N26 antibody was reduced and the safety of the combination therapy of N26 antibody and the irinotecan was subsequently evaluated.

As a result, in 15 patients of 0.5 mg/kg of N26 antibody and the irinotecan administration group, dose limiting toxicity was not found at all in 14 patients, excluding one patient which showed grade 3 colitis, and it was shown that the combination administration of 0.5 mg/kg of N26 antibody and the irinotecan has tolerability. Also in this trial, production of human anti-human antibody was not found in all cases.

Among 16 cases applicable to efficacy evaluation, the patients who was stable (Stable Disease/unchangeable) or better as the best response were 10 patients (63%). In one of the 10 patients, the total of maximum major axes of the target lesion was decreased by 41% in comparison with that of the base line, on week 6 after the start of the combination therapy of N26 antibody and the irinotecan to had Partial Response.

In two of the 9 patients who had stable disease as the best response, a clear reduction of the target lesion was found (on week 6 after the start of the combination therapy of N26 antibody and the irinotecan). One case was 25% reduction of the cancer which metastasized to the lungs, and the other one was 21% of the reduction.

Based on the above, tolerability of the combination therapy of N26 antibody and the irinotecan was confirmed by the first phase part of the first/second combination use clinical test. In addition, although the patients who had the pretreatment histories up to three were allowed to be an object, efficacy of the combination use of N26 antibody with other therapy also came to be bound.

This is the first case in which effect of the combination use of N26 antibody with other therapy was evaluated in human and shows that even in the case of a cancer having resistance to one or two or more of chemotherapies, there is an effect to accelerate involution of the cancer.

While the invention has been described in detail and with reference to specific embodiments thereof, it will be apparent to one skill in the art that various changes and modifications can be made therein without departing from the spirit and scope thereof.

This application is based on U.S. provisional application No. 61/296,180, filed on Jan. 19, 2010, the entire contents of which are incorporated hereinto by reference. All references cited herein are incorporated in their entirety.

What is claimed is:

1. A method for treating colorectal cancer, comprising administering effective amounts of an anti-A33 human antibody and irinotecan in combination to a patient with colorectal cancer.

2. The method according to claim 1, wherein the anti-A33 human antibody and the irinotecan are administered simultaneously or separately.

3. The method according to claims 1, wherein the anti-A33 human antibody and the irinotecan are comprised in a single preparation.

4. The method according to of claims 1, wherein each of the anti-A33 human antibody and the irinotecan is comprised in a separate preparation.

5. The method according to claims 1, wherein the colorectal cancer is at least one of recurrent colorectal cancer and refractory colorectal cancer.

6. The method according to claims 1, wherein the anti-A33 human antibody is administered every week or every other week.

7. The method according to claims 1, wherein the anti-A33 human antibody is administered at a dose of from 0.1 mg/kg to 3 mg/kg.

8. The method according to claims 1, wherein the irinotecan is administered at a dose of 180 mg/kg.

9. The method according to claims 1, wherein the patient with colorectal cancer is a colorectal cancer patient who has not received previous treatment with cetuximab which is an antibody against an epithelial cell growth factor receptor.

10. The method according to 1, wherein the patient with colorectal cancer is a patient with a colorectal cancer who has a mutation in K-ras gene.
